(19) United States
(12) Patent Application Publication
(10) Pub. No.: US 2011/0196290 A1
(43) Pub. Date: Aug. 11, 2011

(54) Jejunal feeding catheter

(76) Inventor: David G. Quinn, Grayslake, IL (US)

(21) Appl. No.: 12/800,588

(22) Filed: May 17, 2010

Related U.S. Application Data
(60) Provisional application No. 61/302,310, filed on Feb. 8, 2010.

(51) Int. Cl. A61M 1/00

(52) U.S. Cl. 604/43

(57) ABSTRACT

An enteral catheter provides access to both the stomach and the deep jejunum for feeding, aspiration and decompression. The catheter includes a triple lumen 16Fr tube that joins to a triple lumen “Y” connector at the proximal end of the tube. The connector serves the three lumens as a source for venting air, for fluid aspiration and for fluid infusion. The catheter includes a gastro/jejunal bolus which provides a large effective recessed port size opening varying from 172 degrees around the circumference of the bolus to a maximum recess circumference of 350 degrees. The gastric aspiration lumen, the jejunal feeding lumen, and the air vent lumen, all connect to the gastro/jejunal or midport connector bolus in the stomach at the distal end of the three lumen tube. The gastric lumen and the air vent lumen both open into the stomach through a common gastric port by the midport bolus. The jejunal lumen in the 16Fr tube communicates with jejunal lumen in the jejunal tube. The midport bolus provides for the attachment of a smaller, round single lumen 8Fr lumen tube that extends into the jejunum and terminates at its distal end with a tip bolus. Both the gastric lumen and the air vent line terminate at the same point side by side into the common gastric port in the midport bolus. The gastric port is recessed to the level of its full internal lumen in the midport bolus, thereby providing a recess for maximum protection against occlusion and maximum area for outflow and inflow. The midport bolus and the jejunal port in the tip bolus include a structural arch protruding radially outwardly therefrom. The arch is effective to prevent the body segment of the bolus from bending and restricting the ports. At the distal end of the jejunal tube, the tip bolus contains an improved port that is recessed to just below the internal radius of the tube lumen to provide maximum protection against occlusion and maximum area for outflow.

### RELATED APPLICATIONS

This application claims priority from U.S. provisional patent application Ser. No. 61/302,310, filed on Feb. 8, 2010. The content of that provisional application is incorporated herein by reference in its entirety.

### FIELD OF INVENTION

This invention relates particularly to catheters for use in administering fluids to body cavities, irrigating the cavities and aspirating the cavities. It relates particularly to catheters and the distal ends thereof that contain the opening(s) for fluid egress or ingress.

### BACKGROUND OF THE INVENTION

The background of this application contains all of the elements of U.S. Pat. No. 7,419,479 and U.S. Pat. No. 7,048,722. The use of Salem sumps in the U.S. for the aspiration of gastric fluid post-surgically in gastrointestinal surgery is well documented. About 10,000 of the tubes are used annually in the U.S.A. 50% of the sump usage is in 18Fr tubes. 25% of the usage is in 16Fr tubes. The remaining usage is spread in decreasing amounts over 14Fr, 12Fr, 10Fr and 9Fr tubes. The vast majority of these tubes utilize polyvinyl chloride as the tube material, commonly referred to as PVC. The use of PVC tubes requires large wall thicknesses and therefore correspondingly large French (Fr) sizes to support internal lumens. The new catheter invention utilizes polyurethane, and therefore smaller French sizes can be utilized. The triple lumen tube described is 16Fr and addresses 75% of the post-surgical enteral feeding market. The existing sump tubes also incorporate conventional, easily clogged inflow ports that are unchanged since the early 1970's. All of the existing post-surgical sump tubes are contraindicated for enteral feeding because of flow port clogging.

The new invention incorporates improved inflow and outflow ports in the midport bolus in the stomach. The port provides for much larger effective and protected port areas to prevent clogging and to maintain aspiration flow. The tube also provides for the feeding of feeding formulas deep into the jejunum through the third lumen with an improved outflow port.

Recent clinical studies have shown that patients in the immediate post-surgical intensive care units should be fed immediately after surgery. It is not possible to immediately enterally feed any of the patients now being given Salem sumps because none of these patients have peristalsis and can, therefore, not empty their stomachs. Stomach contents must be continually aspirated. Studies also show that intensive care patients now receive only 50% of prescribed nutrition. Clinical studies show that if patients are fed immediately after surgery they are released 2.2 days earlier than patients not being fed. Immediate feeding also results in a 55% reduction rate in post-surgical infections.

Many post-surgical patients, including all of those with impaired peristalsis must be fed deep in the jejunum, not the stomach. Clinical studies show that feeding deep in the jejunum does not stimulate the secretion of enzymes into the duodenum. However, deep jejunal feeding does stimulate the secretion of gastric juices, hence the critical need to aspirate the stomach simultaneously and continuously while feeding into the jejunum. Deep jejunal feeding does not stimulate enzyme secretion in the duodenum and therefore deep jejunal feeding must be accomplished with predigested elemental diets that are solutions, not the normal undigested polymeric diets that are emulsions.

Because of the lumen designs and because of the utilization of stronger polyurethane, a midport incorporating an additional third lumen for feeding in the jejunum can be constructed that provides the third lumen for feeding while at the same time providing larger air vent lumens and gastric aspiration lumens per French size than existing Salem sumps.

### SUMMARY OF THE INVENTION

A primary objective of the invention is to provide a new and improved 16Fr three lumen catheter and bolus construction that provides for aspiration of gastric contents, gastric air venting to prevent occlusion during aspiration and the feeding of enteral formulae into the jejunum.

Another objective is to maximize the portion of the three lumen gastro/jejunal bolus that is recessed within the longitudinal circumference of the bolus to provide maximum opportunity for flow egress and ingress through the two bolus ports, gastric and air, that open into the stomach.

Yet another objective is to provide for the minimization of bolus port side walls to provide maximum effective recesses and protected access to the aspiration and air vent ports where the effective recesses from the longitudinal circumference at its maximum recession is approximately 350 degrees (97%), or almost completely surrounding the bolus.

Still another objective of the invention is to provide an effective recess area that varies from a circumference of 172 degrees to 350 degrees.

Another objective of the invention is to provide recessed flow channels in the distal elliptical bolus to provide flow access to the recess 26 from the most distal end of the bolus.

Yet another objective of the invention is to provide a combination access of the middle recess and the flow channels that together form a sphere for flow access to the aspiration port and the air vent port.

Another objective of the invention is to provide a gastric/air vent port whereby both the gastric lumen and the air vent lumen of the triple lumen tube are terminated at the same point at the distal end of the tube so that increases in suction pressure are instantly relieved because of the proximity of aspiration lumen port and the vent line port.

Another objective of the invention is to provide a gastric/air vent port location whereby both the gastric lumen and the air vent lumen of the triple lumen tube are terminated at the same point at the distal end of the tube and share the wall that separates the two lumens so that increases in suction pressure are instantly relieved because of the proximity of aspiration lumen port and the vent line port.

Another objective is to maintain direct access between the terminus of the gastric lumen and the adjacent terminus of the air vent lumen.

Yet another objective is to have the gastric and air vent ports exit from the bolus at a 45 degree angle; the 45 degree angle increasing the effective size of the ports by approximately 25%, further minimizing the possibility of occlusion.

Yet another objective of having the gastric/air vent tube ports exit at a 45 degree angle is to minimize resistance and interface with mucosa during insertion, removal and in-situ.

Yet another objective of having the gastric/air vent tube portion exit at a 45 degree angle is to reinforce the bolster from bending at the point where the recessed portion of the 45 degree angle meets the beginning of the internal ramp leading to the distal elliptical bolus end of the bolus.

Still another objective of the invention is to incorporate a reinforcing arc on the bottom of the bolus to minimize bending.

Another objective of the preferred version is to provide a symmetrical recessed port shape whereby the recessed port has the same shape on both sides with the jejunal lumen providing a central core shape for the bolus.

Another objective of the invention is to provide smooth recessed grooves in the distal elliptical bullet tip of the midport bolus that provide for flow channels in the tip that allows flow communication from the distal end of the bolus to the 350 degree recessed area that communicates with the gastric aspiration lumen and the air vent lumen.

Still another object of the invention is to provide a 16Fr NGJ catheter tube that is the smallest size possible while at the same time providing adequate ingress and egress of fluid and air from both the stomach and the jejunum; the gastric aspiration lumen being the same cross-sectional area as the 18Fr lumens of the commercially available dual lumen gastric sump tubes.

Another objective of the invention is to provide an aspiration lumen that is adequate to serve 75% of gastric aspirations that are now served by a combination of 16Fr (25%) and 18Fr (75%) tubes.

Yet another object of the invention is to provide aspiration lumens that are equal to or larger than the cross-sectional lumens of 18Fr Salem sumps that have cross-sectional areas of approximately 0.0012 in₂.

Another objective is to minimize the total cross-sectional area taken up by the jejunal lumen and the air vent lumen by having them share a communal wall in the 16Fr tube extrusion.

Another object of the invention is to eliminate any recesses or cavities that allow for the collection of any debris in the area of the gastric and air vent ports.

Still another object of the invention is to have the leading distal portion of the gastro/jejunal bolus be formed by an ellipse so to minimize resistance during insertion and removal.

Yet another object of the invention is to have the jejunal lumen in the gastro/jejunal bolus transition to the distal center of the distal elliptical bolus so that the leading edge of the ellipse has an equal amount of exposed space around the exiting jejunal tube so as to present a uniform shape to reduce resistance during insertion.

Still another objective of the invention is to maximize the tip recessed area of the bolus by having the ramp enclosing the jejunal lumen as it moves forward in the bolus begin at a point 0.007″ above the internal radius of the three lumen tube; this internal starting point actually beginning 0.008″ below the bolus radius as the three lumen tube is enclosed by an 0.015″ thickness wall.

Still another objective is provide a inexpensive and effective method of attaching the three lumen tube to the gastro/jejunal bolus by over-molding it to the bolus to the tube, while the distal tip bolus of the single lumen jejunal tube is glued to the distal single lumen bolus.

Yet another objective of the invention is to provide an improved distal jejunal bolus tip that incorporates the enlarged access features of the gastro/jejunal bolus.

In general, the objectives of the invention are similar to those of Quinn patents U.S. Pat. No. 7,419,479, and U.S. Pat. No. 7,048,722, but with significant new features and advantages. The main new advantages are embodied in the over-mold of the midport bolus to the multi-lumen tube, which greatly increases the size of the recessed areas in the bolus that protect the gastric and air vent ports for unimpeded ingress and egress of flow.

### BRIEF DESCRIPTION OF THE DRAWINGS

The invention, including its construction and method of operation, is illustrated more or less diagrammatically in the drawings, in which:

FIG. 1 is a side view of the entire gastro/jejunal catheter, including a three-port “Y” connector, the transitional gastric bolus and the jejunal bolus;

FIG. 2 is a side plan view of a gastro/jejunal catheter showing the gastro/jejunal bolus;

FIG. 3 is a side longitudinal sectional view of FIG. 2 taken through the gastro/jejunal bolus of FIG. 4;

FIG. 4 is a top plan view of the gastric bolus of FIG. 2;

FIG. 5 is a bottom plan view of the gastric bolus of FIG. 2;

FIG. 6 is the same side plan view of the gastric bolus as shown in FIG. 2;

FIGS. 7 through 19 are cross-sectional views taken along lines 7-7, 8-8, 9-9, 10-10, 11-11, 12-12, 13-13, 14-14, 15-15, 16-16, 17-17, 18-18, and 19-19 of FIG. 6;

FIG. 20 is a cross-sectional view of the aspiration lumen as shown in FIG. 7 that further shows the cross-sectional area of the lumen in inches squared;

FIG. 21 is a cross-sectional view of the jejunal lumen as shown in FIG. 7 that further shows the cross-sectional area of the lumen in inches squared;

FIG. 22 is a cross-sectional view of the air vent lumen as shown in FIG. 7 that further shows the cross-sectional area of the lumen in inches squared;

FIG. 23 is a demonstration cross-sectional view of a 13Fr round single tube that has the same cross-sectional area as the invention's aspiration lumen.

FIG. 24 is a cross-sectional view taken at 24-24 of FIG. 2 at an angle of 45 degrees to the longitudinal plane of the gastro/jejunal bolus that further shows the increased cross-sectional area of the aspiration lumen in inches squared;

FIG. 25 is a cross-sectional view taken at 23-23 of FIG. 2 at an angle of 45 degrees to the longitudinal plane of the gastro/jejunal bolus that further shows the increased cross-sectional area of the jejunal lumen in inches squared; and

FIG. 26 is a cross-sectional view taken at 23-23 of FIG. 2 at an angle of 45 degrees to the longitudinal plane of the gastro/jejunal bolus that further shows the cross-sectional area of the air vent lumen in inches squared.

### LIST OF DRAWING REFERENCE NUMBERS

10 gastro/jejunal catheter;
14 jejunal bolster;
16 gastro/jejunal bolster;
18 triple port “Y”;
20 triple lumen gastro/jejunal tube;
21 ramp;
22 lowest level of recessed groove of air vent port and gastric port;
23 over-molding and socket area;
24 socket for attachment of jejunal tube;
25 leading edge of gastric lumen 38;
26 recess created by side ramp;
27 most-recessed portion of side recess 26;
28 radius of triple lumen gastro/jejunal tube 20;
29 beginning point of ramp 21;
30 flow channel;
31 ramp meets ellipse;
32 single lumen jejunal tube;
33 distal elliptical portion of gastro/jejunal bolus gastro/jejunal bolus 16;
34 distal jejunal bolus tip;
36 reinforcing arc;
38 gastric aspiration lumen;
39 communal wall shared by gastric lumen and air lumen;
40 jejunal feeding lumen;
41 radius of three-lumen tube;
42 cross-section of the septum separating gastric lumen 38 and jejunal lumen 40;
48 air vent lumen; and
52 jejunal lumen in jejunal tube.

The preferred embodiments Section, which follows, uses the descriptions of the foregoing invention elements with reference numbers.

### DESCRIPTION OF THE PREFERRED EMBODIMENTS

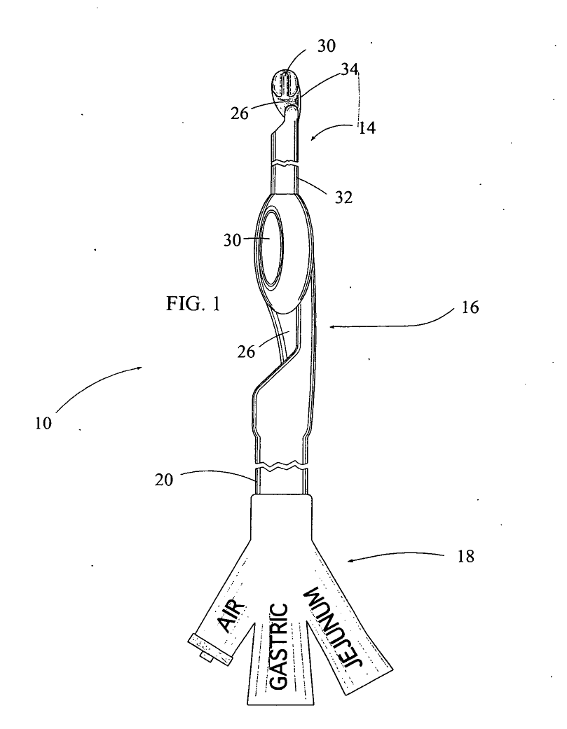

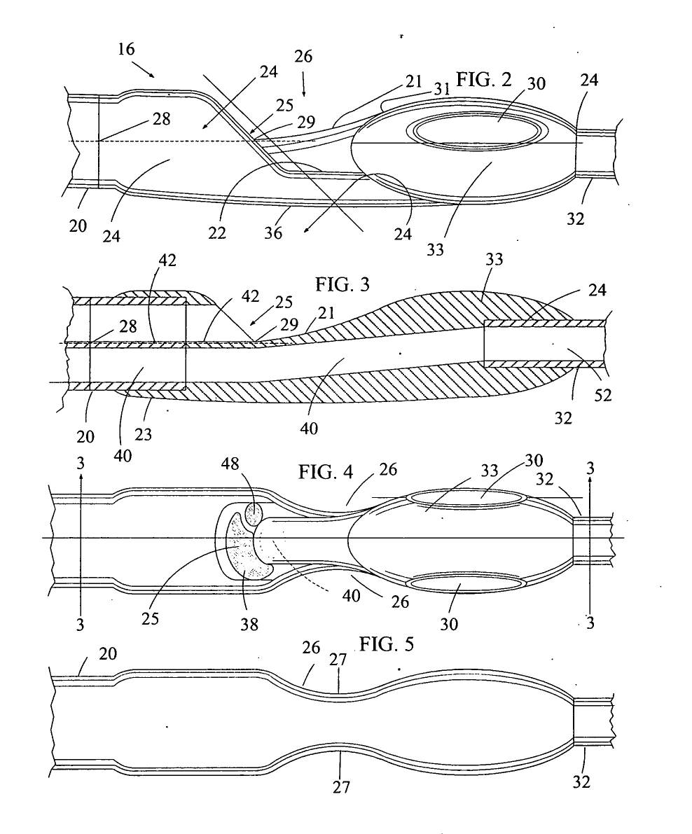

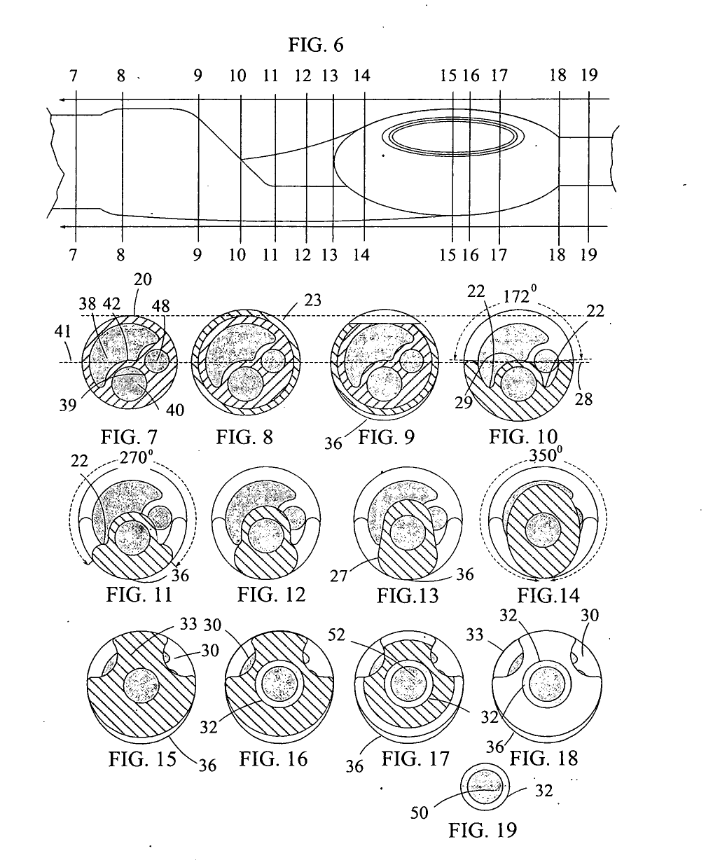

Reference is now made to the drawings, especially FIGS. 1, 2, 3, 4, and 5. In FIG. 1 the three major invention components are shown. In FIG. 1 the entire assembly 10 consisting of the triple lumen “Y” connector 18, the transitional bolus assembly 16 and the jejunal tip 14 is shown. In FIG. 2 the 16Fr triple lumen tube is shown at 20. This tube 20 for adult or juvenile uses can be either 42″ or 36″ long, depending on the size of the patient. The goal is to place the bolus 16 in close proximity to the pylorus so that the stomach can be aspirated at its emptying point. The tube size disclosed is a 16Fr triple lumen catheter with an outside diameter of 0.216.″ The midport bolus is shown at 16 and is 1.040″ in length. The over-molded bolus is 0.246″ OD. The single lumen jejunal tube 32 exiting the distal end of bolus 16 is an 8Fr single lumen tube with a outside diameter (OD) of 0.124″ and an ID of 0.080″. Its length is 42″ or 36″. Therefore, the full length of the tube is either 76″ or 82.″

In FIG. 2 the socket area 23 encloses triple lumen tube 20 where it is over-molded on the 20 tube. The top of the ramp 21 leading from gastric aspiration lumen 38 leads to distal end bolus tip 34. The bottom-recessed groove level of air vent port 48 is shown at 22. The continuation of side recess area is shown at 26. As shown in FIGS. 2, 3, 4 and 5 the recess 26 continues the full side of the bolus 16. 25 is the overall gastric lumen 38 that is formed by both the gastric lumen 40 and the air lumen 48 once they exit the over-molding area 23. The reinforcing arc is shown at 36.

FIGS. 2 and 3 show the ramp 21 enclosing jejunal lumen 40 and extending from its beginning 29 where the ramp meets the 45 degree termination of gastric port lumen 38 and air vent port lumen 48. The ramp terminates where it blends with the distal elliptical bolus at 31.

Flow channel 30 is shown in FIGS. 2 & 3. These channels are on both sides of the ellipse 33. In FIG. 1. The jejunal bolus is shown at 34. This bolus: incorporates flow channels 30 and a flow recess 26 that are also incorporated in the gastro/jejunal bolus.

In FIG. 3 the jejunal lumen 40 exits the triple lumen tube 20 and extends to a point 29 where the lumen blends into an upward path until it encounters the tube socket 24. At this point it straightens again into a path that is parallel to the long axis of the bolus 16 and becomes lumen 52 of 8Fr jejunal tube 32. This transition allows the passage of guidewires or stylets.

FIG. 3 shows a cross-section of the bolus at section 3 of FIG. 4. 42 is the cross-section of the central septum separating gastric lumen 38 and jejunal lumen 40. 52 is the jejunal lumen in the tube 32, that is the jejunal lumen in the 8Fr single lumen jejunal line 32.

FIG. 4 shows a top view of the bolus. The recessed area of the bolus blends from 22 and is shown at 26. FIG. 5 shows the bottom of the bolus. The recess on the side of the bolus is shown at 26.

FIGS. 7-19 show cross-sections in FIG. 6. FIG. 7 is a cross-section of the triple lumen tube 40. FIG. 8 is a cross-section of the over-molded wall of 0.015″ covering and securing the tube 20. FIG. 9 shows the point where the 45 degree slope begins. FIG. 10 shows the face of the 45 degree slope and the cross-section of the bolus beneath it at 22. FIG. 10 also shows the recessed circumference of 172 degrees where the recess 26 begins.

FIGS. 10-14 show the air-line 48 bottom portion 22 as it transitions distally. FIG. 11 shows the transition of the recess 26 where it forms a 270° recess on the side of the bolus. The reinforcing arc is seen at 36. This thickened arc adds stiffness to the bolus and prevents it from kinking. Its function is also incorporated in the jejunal tip bolus 34 shown in FIG. 1.

Now referring to FIGS. 7-19. All of these figures utilize the cross-sections of multi-lumen tube 20 and the bolus 16. FIG. 7 shows the 270° effective port opening and its symmetrical shape with the jejunal lumen being the midpoint core of the bolus 16. In FIG. 14 the bolus cross-section achieves its maximum recessed circumference of 350 degrees.

Now referring to FIGS. 15 through 18. FIG. 15 shows reinforcing arc 36 and shows the flow channels 30. Note that jejunal lumen 40 is in its ascending segment. In FIG. 16 the ascending jejunal lumen 40 meets the parallel tube socket 24 and the tube 20. In FIGS. 17 and 18 the parallel jejunal lumen 52 continues though tube 32 and finally exits the bolus as an 8Fr tube in FIG. 19.

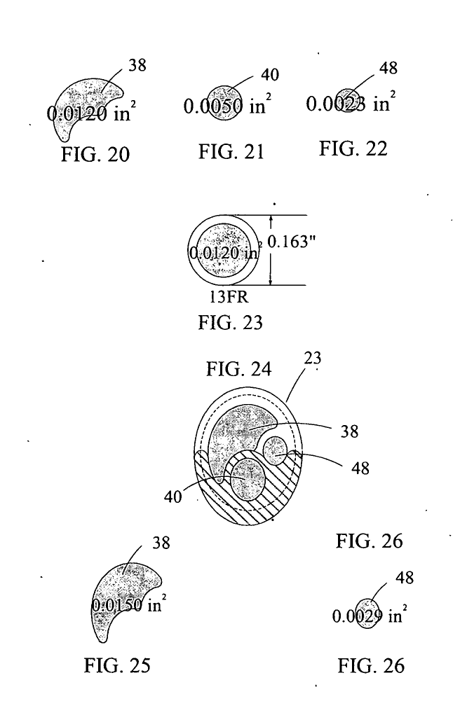

Referring to FIGS. 20, 21, 22 and 23. FIG. 20 is a cross-sectional view of the aspiration lumen 38 showing graphically the cross-sectional area of 0.0120 square inches, which is slightly larger than existing gastric aspiration lumens utilized in dual lumen tubes. FIG. 23 is a demonstration round lumen tube with the same cross-sectional area as FIG. 20. This lumen is equal to 13Fr single lumen tube. The largest lumen cross-sectional area of 0.008 is utilized in both the 10Fr and 12Fr most commonly used, enteral feeding tubes. These tubes are used for both feeding and gastric aspiration. However, the lumen 38 is 150% larger and provides more than adequate cross-sectional area for aspiration.

FIG. 21 shows the 0.005″ cross-sectional area of the jejunal lumen 40. This lumen size is identical to the area of an 8Fr enteral feeding tube that is the most widely utilized size. In the case of the new bolus, feeding will be deep in the jejunum, thereby requiring a longer tube than the standard gastric length enteral feeding tube. However, in deep jejunal feeding less viscous elemental solution diets are employed rather than the viscous polymeric emulsion diets utilized in gastric feeding. Therefore an 8Fr tube is adequate for the invention.

Now referring to FIG. 22, the air vent lumen cross-section is 0.0023″ squared, which is the same size as a 6Fr enteral feeding tube and is more than adequate to provide air to the stomach.

FIG. 24 is a 45 degree view taken through section 24-24 of FIG. 2. The 45 degree angle increases the effective size of the gastric port 38 and the air vent port 48 by approximately 25%. This increase in size is important in helping to prevent occlusion by either debris or gastric mucosa. However, the increased port size does not effect, in any way, the flow rate which is controlled by the cross-sectional area of the tubing at 90 degrees.

FIG. 25 shows the increased cross-sectional area of the aspiration'port, and FIG. 26 shows the increased cross-sectional area of air vent lumen.

While preferred embodiments of the invention have been described, it should be understood that the invention is not so limited, and modifications may be made without departing from the invention. The scope of the invention is defined by the appended claims, and all devices and methods that come within the meaning of the claims, either literally or by equivalence, are intended to be embraced therein.

The invention claimed is:

1. A catheter for delivering fluid into, or aspirating fluid out of, a body cavity or cavities, comprising:
a) a multiple lumen tube containing at least a first lumen, a second lumen and a third lumen and having a proximal end and a distal end, said multiple lumen tube containing a septum separating said first lumen and second lumens and a septum separating first and second lumen from said third lumen;
b) first and third lumens being formed so that first lumen and third lumen are the same length and open at the same distal point;
c) said second lumen being formed so that said lumen is longer than said first and second lumen and both said septums terminate at the distal ends of first and third lumens;
d) said second lumen terminates at a predetermined distance from where said first and third lumens open at said distal end of said multiple lumen tube;
e) a first bolus having a nose end and a connector end and an axial passage therethrough;
said first bolus being formed independently of said multiple lumen tube and said distal end of multiple lumen tube being over-molded or seated over axial passages of said first, second and third lumens;
g) a single lumen catheter tube separates from said multiple lumen tube and seated in axial passage of first said bolus at its nose end and a port in its distal end; and
h) a second bolus on the distal end of said single lumen catheter tube;
i) said port in said distal end of said single lumen tube being formed in the side of the single lumen bolus.

2. The catheter of claim 1 further characterized in that: a) said second bolus has a nose end which is bullet shaped and which is smooth but has grooves and recessed areas to assist in flow and access.

3. A catheter, comprising:
a) a multiple lumen catheter tube, containing first, second and third lumens;
b) a bolus formed independently of said multiple lumen tube, said bolus being connected to said distal end of said tube, said bolus forming at least a portion of a each of a first lumen port extending radially of said catheter over a substantially recessed outer wall of said tube;
c) said first port communicating with first said lumen, and a second port from lumen three communicating with said first port;
d) said first lumen extending to an opening at a predetermined distance from said distance from said distal end of multiple tube; and
e) said bolus including an attachment section fastened to said septum where it comprises and outer wall and has a rear face defining a ramp including a surface inclined at an angle to said septum.

4. The catheter of claim 3 further characterized in that:
a) said ramp extends rearwardly to an intersection with first lumen opening.

5. The catheter of claim 3 further characterized is that:
a) said catheter tube contains a third lumen.

6. The catheter of claim 3 further characterized in that:
a) the third lumen forms a recessed ramp on the side of the first bolus;
b) the said ramp transitions distally to the end of the bolus.

7. The catheter of claim 3 is further characterized in that:
a) the recessed top ramp of the first lumen and the side recessed ramp of the third lumen communicate with each other;
b) the said communicated recessed ramp form a recessed ramp for flow and aspiration around 350° of the circumference of the first bolus.

8. The catheter of claim 3 is further characterized in that:
a) the first and third lumens of the multiple lumen catheter tube terminate at the same distal point;
b) both said first and third lumens are adjacent to each other;
c) both said first and third lumens communicate with the 350° top and side recessed ramp.

9. The catheter if claim 3 is further characterized in that:
a) the 350° recessed ramp in the first bolus surrounds the second lumen that extends to the distal end of the single lumen tube;
b) the portion of the second lumen contained in the first bolus transitions from semi “D” shape to a circular shape at the distal end if the said bolus.

10. The catheter claim 3 is further characterized in that:
a) all of the attachment intersections of the four points whereby the internal lumen septums intersect with the outer main tube wall are at approximately 45° tangents to the outer wall.
